(19) United States
(12) Patent Application Publication
(10) Pub. No.: US 2012/0252012 A1
(43) Pub. Date: Oct. 4, 2012

(54) METHOD FOR DETECTING PROKARYOTIC DNA FROM A FECES SAMPLE

(75) Inventors: Fabrice Armougom, Nans Les Pins (FR); Michel Drancourt, Marseille (FR); Mireille Henry-Mary, Greasque (FR); Didier Raoult, Marseille (FR)

(73) Assignees: ASSISTANCE PUBLIQUE - HOPITAUX DE MARSEILLE, Marseille Cedex 05 (FR); UNIVERSITE DE LA MEDITERRANEE (AIX-MARSEILLE II), Marseille Cedex 07 (FR)

(21) Appl. No.: 13/320,343

(22) PCT Filed: Apr. 30, 2010
(86) PCT No.: PCT/FR10/50824

§ 371 Date: Jan. 30, 2012

(30) Foreign Application Priority Data
May 14, 2009 (FR) 0953207

(51) Int. Cl. C12Q 1/68; G01N 21/64

(52) U.S. Cl. 435/6.11; 435/6.15; 435/6.12

(57) ABSTRACT

The present invention concerns a method of detection and preferably of quantification of DNA, preferably comprising prokaryotic DNA, extracted from a stool sample of an individual, especially for the molecular determination of the composition of the intestinal flora in the stool. According to the invention, one controls the quality of the DNA extraction by verifying whether one detects a specific DNA of Methanobrevibacter smithii and one performs the quantification of said specific prokaryotic DNA for Archae Methanobrevibacter smithii, for the bacterial genus Lactobacillus, for the phylum Bacteroidetes and for the phylum Firmicutes, respectively, to provide a diagnosis and/or monitoring of the weight status of an individual.

The present invention provides a method for carrying out the extraction of prokaryotic DNA in stools.

The present invention concerns a method of detection and preferably of quantification of DNA, preferably comprising prokaryotic DNA, extracted from a stool sample of an individual, especially for the molecular determination of the composition of the intestinal flora in the stool.

The present invention also concerns a kit for detection and quantification of DNA that can be used to implement a method of detection and quantification of DNA according to the invention.

It is useful to be able to determine the composition of the intestinal flora (termed here the microbiota or intestinal microbiota) in man, because this flora is involved in the physiological processes of digestion of food and drink, and its role is suspected or confirmed in various pathological digestive and nondigestive processes.

For example, the role of the intestinal microbiota in obesity is a topic of interest [Turnbaugh P J et al.—A core gut microbiome in obesity an lean twins. Nature 2009; 457:480-4]. In fact, the weight condition of an individual is the result of the number of calories consumed (role of nutrition), the number of calories extracted during digestion (role of the intestinal microflora), the number of calories stockpiled (role of the adipose tissues), and the number of calories expended (role of physical exertion). It is now known that all the microbial species of the intestinal microbiota do not have the same ability to extract the calories brought in by nutrition, certain species or groups of species (called here phyla) being able to convert by a cascade of biochemical reactions the calories contained in food and drink better than other species or other phyla. For example, there is much evidence that the composition of the intestinal microbiota in the mouse influences weight gain, even in mice that do not have a genetic basis for obesity (Leptine -) [Turnbaugh P J et al.—An obesity-associated gut microbiome with increased capacity for energy harvest. Nature 2006; 444:1027-31; Turnbaugh P J et al.—Diet-induced obesity is linked to marked but reversible alterations in the mouse distel gut microbiome. Cell Host Microbe 2008.3:213-223; Bäckhed F. et al.—The gut microbiota as an environmental factor that regulates fat storage. Proc. Natl. Acad. Sci. USA 2004; 101: 15718-15723; Ley R E et al.—Human gut microbes associated with obesity. Nature 2006; 444:1022-3]. In man, experimental work has not been possible and information as to the role of the intestinal microbiota in the weight condition of individuals is based essentially on observation of the composition of the intestinal microbiota in different circumstances. A first work has shown that obese individuals had a skewing of the ratio of bacteria of the phylum Firmicutes with respect to the bacteria of the Bacteroidetes (Firmicutes/Bacteroidetes or F/B ratio), as compared to the control subjects, due primarily to the decrease in Bacteroidetes in obese individuals [Ley R E et al.—Human gut microbes associated with obesity. Nature 2006; 444:1022-3]. On the other hand, many studies had shown that Bacteroides thetaiotaomicron caused an increase in the conversion into calories in xenobiotic mice [Samuel B S et al.—A humanized gnotobiotic mouse model of host-archaeal-bacterial mutualism. Proc Natl Acad Sci USA 2006, 103: 10011-10016]. These two findings are not incompatible, since the decrease in the genus Bacteroidetes does not necessarily result in a decrease in the species Bacteroides thetaiotaomicron, but rather there might be a drastic drop in other Bacteroidetes and a relative rise in Bacteroides thetaiotaomicron.

Moreover, the inventors have tested the primers used by the various authors who have published on this subject and observed that these primers are not absolutely specific for B. thetaiotaomicron and they amplify other species of the genus Bacteroides. The same work has shown that correction of overweight by a low-calorie or low-carbohydrate diet caused a progressive modification of the microbiota in the course of a year, it tending to become comparable to that of thin persons [Ley R E et al.—Human gut microbes associated with obesity. Nature 2006; 444:1022-3].

More recently, it was shown that microorganisms belonging to the realm of the Archae, more precisely the order Methanobacteriales, were more numerous in obese individuals than in the controls or in individuals having undergone a gastric by-pass surgery for obesity. However, the precise determination of the genera or species of Archae associated with obesity was not done in this work.

In particular, the authors did not determine precisely which of the two species of methanogenic Archae known in the intestinal microbiota, Methanobrevibacter smithii and Methanosphaera stadmanae, was more specifically associated with the microbiota of obese individuals [Zhang H. et al.—Human gut microbiota in obesity and after gastric bypass. Proc. Natl. Acad. Sci. 2009; 106:2365-70].

Other examples of pathologies during which the intestinal microbiota is known or suspected to play a role involve the genesis of colon cancer [Piqué J M et al.—Methane production and colon cancer. Gastroentorology 1984; 87 :601-5] and other chronic inflammatory digestive pathologies [Peled Y. et al.—Factors affecting methane production in humans. Gastrointestinal diseases and alterations of colonic flora—Dig. Dis. Sci. 1987; 32: 267-71; Waever G A et al.—Incidence of methanogenic bacteria in sigmoidoscopy population: an association of methanogenic bacteria in diverticulosis. Gut 1986; 27:698-704].

These various examples illustrate the interest of both research laboratories and medical and veterinary diagnostic laboratories in being able to routinely determine the composition of the intestinal microbiota, and especially the relative proportion relative of a particular species of microorganism or a particular phylum of microorganisms. In the example of an individual's weight condition, it is useful to be able to determine by simple microbial cartography of the stool whether the individual is predisposed to a pathological emaciation or an obesity, which might not be properly reflected by the simple measurement of body weight at a given time. Likewise, it is useful to be able to measure objectively the evolution of this cartography during the treatment of the emaciation or the obesity so as to analyze the efficacy of the treatment and obtain a forecast of the success of the therapy before the measurement of body weight reaches the goal of the treatment.

Even so, the methods currently published for the analysis of the intestinal microbiota do not allow a routine use, either in research laboratories or in medical or veterinary diagnostic laboratories. This is illustrated by the fact that the published works deal with a relatively small number of individuals studied, between 9 [Zhang H. et al.—Human gut microbiota in obesity and after gastric bypass. Proc. Natl. Acad. Sci. 2009; 106 :2365-70] and 154 [Turnbaugh P J et al. A core gut microbiome in obese and lean twins. Nature 2009; 457:480-4]. In fact, the works done so far on the microbiota have all used PCR amplification of the universal gene 16S ribosomal RNA, followed either by cloning and sequencing by the Sangers technique of a large number of clones (on the order of 100 clones), or a high-speed pyrosequencing technique on the platform 454 Life Sciences—Roche [Zhang H. et al.—Human gut microbiota in obesity and after gastric bypass. Proc. Natl. Acad. Sci. 2009; 106 :2365-70]. These techniques certainly cannot be used routinely, due to their cost, their cumbersome use, and the time it takes to get results, being several weeks. Furthermore, the various works carried out produce variable results for the control subjects among the different studies. This is due in part to the absence of an internal quality control, guaranteeing the quality of extraction of the nucleic acids from the stool sample and the absence of an internal reference in the sample that could serve to normalize the samples and more easily compare the experimental results obtained in different individuals.

The methods used thus far are not reliable in terms of extraction and quantification of the prokaryotic DNA of the stool, and they are not practically adapted to a use in routine analysis, whether in a biological analysis laboratory or a research laboratory.

In SCANLAN et al. BMC Microbiology 2008 Vol 8, No. 1, pages 1471-2181, they describe the extraction of DNA from stool samples and the detection of the mcrA gene of M. smithii in only 24% to 48% of the samples tested, depending on the type of individual. The mcrA gene is not a gene specific to M. smithii but is found in other methanogenic archae.

In RIDLON et al., Clinica Chimica Acta 357 (2005 55-64), the SSU rDNA gene of M. smithii is detected and quantified in a single sample and neither is it specific to M. smithii but is found in all other methanogenic archae. On the other hand, the DNA extraction protocol used does not include a chemical lysis, or a thermal lysis.

The inventors have found that the extraction methods used in the prior art to extract DNA from stools were not effective enough to produce reliable findings in terms of quantification, especially when one is trying to quantify prokaryotes of thick-walled Archae type.

This has led the inventors to develop a new method of extraction involving at least a mechanical lysis, prior to a chemical lysis and, more precisely, the performance of a second mechanical lysis after the first chemical/enzymatic lysis.

In parallel and in addition, the inventors have developed tools to carry out quantitative PCRs, with sets of primers and probes specific to a series of potentially interesting candidates in the analysis of the intestinal flora, taking into account certain papers from the literature, especially as regards the Bacteroidetes and Firmicutes, but also taking into account the personal analysis of the inventors, especially as regards Lactobacillus.

The inventors have developed a method of quantification of certain species of microorganisms and certain phyla of microorganisms, starting from a stool sample, of routine application in research laboratories and medical and veterinary diagnostic laboratories, and including an internal quality marker.

More particularly, the inventors have created a method of molecular quantification of bacteria of the phylum Firmicutes, and specifically the bacteria of the genus Lactobacillus, and bacteria of the phylum Bacteroidetes. The choice of the phyla Firmicutes and Bacteroidetes has been explained above. The choice of the genus Lactobacillus, which is a genus of the phylum Firmicutes, is justified by the fact that growth promoters and particularly Lactobacillus were associated in the chicken with a very substantial weight gain, especially in those that were supplemented with Lactobacillus during their early life [Khan M et al.—Growth-promoting effects of single-dose intragastrically administered probiotics in chickens. Br Poult Sci 2007, 48: 732-735]. The inventors have inferred from this that this bacterial genus could also help in regulating the weight condition of an individual, even though no information had been published regarding humans, despite major conclusions that could be drawn in view of the fact that Lactobacillus is present in the food products coming from the agrobusiness industries.

For this, the inventors developed a technique of determination and quantification of each of the species/phyla of interest by quantitative PCR, using Archae Methanobrevibacter smithii as an indicator of the quality of the extraction of the prokaryotic DNA from the stool sample.

The inventors formed the hypothesis that Methanobrevibacter smithii, an Archae microbe very difficult to extract, should be present in all human stool samples, due to the fact of its role in the detoxification of products resulting from the transformation of foods in the intestines by synthesis of methane, and thus its identification and quantification should constitute a positive control of the proper performance of the extraction and quantification of the sample.

The inventors thus first of all perfected a technique of extraction of the DNA of microorganisms from stools, which allowed them to verify by the constant presence of Archae Methanobrevibacter smithii in human stools the quality of this extraction procedure. Next, they perfected a method of quantification of the microorganisms of interest, with a view to developing a routine protocol making it possible to quantify, from a stool sample of humans or animals, the presence of microorganisms of interest, especially Methanobrevibacter smithii, Lactobacillus spp., Firmicutes and Bacteroidetes.

The present invention thus provides a very efficient protocol for extraction of prokaryotic DNA (Bacteria and Archaea) and relative quantification of the intestinal prokaryotic flora, one that is exhaustive, efficient, reproducible and easy to carry out routinely in the laboratory in order to facilitate the analysis of a large number of samples for both diagnostic and epidemiological purposes.

If order for the quantification tool of the intestinal bacterial flora to be efficient, sensitive and reproducible, it is necessary for the upstream technique used for the extraction of prokaryotic DNA (Bacteria and Archaea) from stools to enable the extraction of the entirety of the DNA.

To do this, the inventors perfected a technique of extraction of the DNA by alternating mechanical lysis and enzymatic lysis, which they controlled by introducing into a reference stool known quantities of prokaryotes (Bacteria, Archaea) whose DNA extraction is difficult because of the presence of a particularly thick cell wall, such as Methanobrevibacter smithii. In fact, a reliable quantification is only possible with an optimal extraction of the DNA. The inventors optimized all the phases of extraction of the lysis of bacteria, down to the choice of the method of extraction, whether it be manual or automatic, taking into account the effectiveness and the reproducibility of the method. Finally, the inventors compared the extraction technique found with that used by the main authors who have published on this subject, as described in example 1.

The method of extraction of DNA, as well as the tools for real-time PCR quantification developed by the inventors, allowed them to confirm that Methanobrevibacter smithii was present in 100% of individuals, in very variable levels, especially depending on their weight condition.

The inventors concluded from this that it was of interest to systematically analyze stools for presence of Methanobrevibacter smithii, at least as a quality control for the extraction of the DNA from the stools in the tested sample.

Likewise, it was also discovered that the other methanogenic prokaryote, Methanosphaera stadmanae, was present only in the stools of 38% of individuals. It was this additional fact that also led the inventors to propose the detection of Methanobrevibacter smithii as a reference for a good extraction of the DNA.

Thus, the present invention provides a method of detection and preferably of quantification of DNA, possibly comprising prokaryotic DNA, extracted from a stool sample of an individual, characterized in that one controls on the quality of the extraction of the DNA by verifying whether a specific DNA of Methanobrevibacter smithii is detected, and preferably quantified at a rate of at least 10⁴ organisms of M. smithii ml of said stool sample.

Obviously, one will not consider the results from detection and quantification of any DNA extracted from said sample if the quality control for the extraction is negative, that is, if one does not detect said specific sequence of M. smithii in the DNA extracted from said sample.

More particularly, one quantifies the DNA copy number of at least one DNA sequence specific for Methanobrevibacter smithii by quantitative PCR, chosen from among the following specific sequences:
a sequence taken from the gene taken from the 16S gene of the ribosomal RNA,

SEQ. ID. N^{o} 1 = 5′-CCGGGTATCTAATCCGGTTCGCGCCCCTAGCTTTCGTCCCTCACCGTC
AGAATCGTTCCAGTCAGACGCCTTCGCAACAGGCGGTCCTCCCAGGATTACAGAATTTCACCTCTAC
CCTGGGAG -3′,
and,

a sequence taken from the gene rpoB,

SEQ. ID. N^{o} 5 = 5′-AAGGGATTTGCACCCAACACAATTTGGTAAGATTTGTCCGAATGAAAC
CCCAGAGGGTCCTAACTGTGGTC -3′

Even more particularly, one quantifies the number of copies of said DNA specific for Methanobrevibacter smithii by quantitative PCR in real time, involving PCR type enzymatic coamplification of a sequence specific for Methanobrevibacter smithii contained on the one hand in said DNA extracted from the stool sample and on the other hand in a synthetic sample of DNA fragments serving as a standard for quantification of the DNA, said sequence specific to Methanobrevibacter smithii being chosen among the following sequences or their complementary sequences:
a sequence of the gene 16S RNA amplifiable by the following primer sequences:

Sense primer, SEQ. ID. N^{o} 2:
5′- CCGGGTATCTAATCCGGTTC -3′, et
Antisense primer, SEQ. ID. N^{o} 3:
5′- CTCCCAGGGTAGAGGTGAAA-3′,

and
a sequence of the gene rpoB amplified by the following sequence primers:

Sense primer, SEQ. ID. N^{o} 6:
5′- AAGGGATTTGCACCCAACAC -3′,
and
Antisense primers, SEQ. ID. N^{o} 7:
5′-GACCACAGTTAGGACCCTCTGG -3′.

Preferably, one quantifies at least two specific sequences of Methanobrevibacter smithii taken respectively from the ribosomal RNA gene 16S and from the gene rpoB. The inventors have in fact observed that a detection of Methanobrevibacter smithii in 100% of individuals requires performing the detection on two genes, preferably, the quantity of M. smithii being quantified at a level of at least 10⁴ organisms of M. smithii ml in said stool sample. In fact, a quantification of 10⁴ organisms/ml of stool sample corresponds to the sensitivity threshold of detection below which one does not detect any DNA with the primers SEQ. ID. No. 6 and 7 for the gene rpoB of M. smithii and the primers SEQ ID. No. 2 and 3 for the RNA gene 16S of M. smithii.

Preferably, one performs reactions of amplification and quantification by PCR in Real Time, making use of specific hydrolysis probes for Methanobrevibacter smithii.

The technique of Real Time PCR consists in a classical PCR making use of direct and inverse sequence primers, and involves a detection of the amplified product based on measuring the emission of fluorescence proportional to the quantity of genes amplified with a so-called “hydrolysis” probe. For this, said probe is marked by an emitter of fluorescence or fluorophore at 5′ and an agent blocking the emission of fluorescence at 3′. This blocking agent absorbs the fluorescence emitted when the fluorophore and the blocking agent are close together. When the fluorophore and the blocking agent are separated, the emission of fluorescence is no longer absorbed by the blocking agent. During its passage, Taq polymerase produces a hydrolysis of the probe and thus a releasing of nucleotides and the fluorophore in solution. The emission of fluorescence will thus be proportional to the number of amplifiates. The principle of real-time PCR is based on the ability of Taq polymerase during the elongation phase to hydrolyze a probe hybridized on the DNA being copied, this hydrolysis enabling the emission of fluorescence, which enables a quantification. During the same reaction, one can quantify two different targets by introducing into the reaction mixture two primers and one probe directed at a first target, and two other primers and probe directed at another target. The two probes being marked with different fluorophores.

Preferably again, one uses a large synthetic DNA fragment serving as reference standard for the quantification of the DNA, said large synthetic DNA fragment bringing together said specific sequences of each of said bacteria or prokaryotic species whose concentrations are quantified. The presence of several molecular targets on the same nucleic fragment lets one quantify different targets in the same sample and co-quantify them in homogeneous manner, the quantification making use of the same calibration range for several molecular species and allowing the comparison of the effectiveness of different PCR reactions with each other and from one determination to another over the course of time, and it avoids the bias associated with the positive control.

By “DNA fragment” is meant a fragment of DNA or oligonucleotide whose sequences are written hereinafter in the direction 5′-3′.

More particularly, one carries out a PCR reaction of amplification and quantification in real time making use of specific hydrolysis probes for each of said specific sequences of Methanobrevibacter smithii, namely:

1) for the sequence of the ribosomal RNA gene 16S,

SEQ. ID N^{o} 4 = 5′-CCGTCAGAATCGTTCCAGTCAG -3′,

and

2) for the sequence of the gene rpoB,

SEQ. ID N^{O}8 = 5′-ATTTGGTAAGATTTGTCCGAATG-3′,

and
a large fragment of synthetic DNA, serving as standard for quantification of the DNA, said large fragment of synthetic DNA bringing together said specific sequences, preferably in the form of a plasmid, with a plurality of samples of large synthetic DNA fragment of different known concentrations.

The sequences of the molecular targets amplified have the following structure, with the primer sequences flanked and the probe sequences flanked and italicized:

For SEQ. ID. N^{o} 1:
CCGGGTATCTAATCCGGTTCGCGCCCCTAGCTTTCGTCCCTCACCGTCA
GAATCGTTCCAGTCAGACGCCTTCGCAACAGGCGGTCCTCCCAGGATTA
CAGAATTTCACCTCTACCCTGGGAG,
and,
For SEQ. ID. N^{o} 5:
AAGGGATTTGCACCCAACACAATTTGGTAAGATTTGTCCGAATGAAACC
CCAGAGGGTCCTAACTGTGGTC

The inventors have thus developed a tool for quantification having as its targets sequences able to specifically and reliably quantity: (1)—the Firmicutes (Fi), (2)—the Bacteroidetes (Ba), (3)—the Lactobacillus (La) in order to determine by real-time quantitative PCR the relative composition of the intestinal microbiota of obese individuals, thin individuals, and individuals having a psychological anorexia.

More precisely, in one method according to the invention, one also jointly quantifies in said DNA extracted from said stool sample at least one specific sequence chosen from among:

1) a specific consensus sequence of the phylum Bacteroidetes, and

2) a consensus sequence specific of bacteria of the genus Lactobacillus, and

3) a specific consensus sequence specific of the phylum Firmicutes.

By “specific sequence” is meant a sequence of the genome of said species Methanobrevibacter smithii, of said Lactobacillus gene or said phylum Bacteroidetes or Firmicutes, that is not found in any other genome of living organisms or microorganisms.

By “specific consensus sequence” is meant here a sequence of the genome of said species Methanobrevibacter smithii, of said Lactobacillus gene or of said phylum Bacteroidetes or phylum Firmicutes, that is found in all said strains of said species, said genus or said phylum and not found in any other genome of living organisms or microorganisms.

As for the bacteria Firmicutes, the inventors have developed a set of original primers and probes taken from the ribosomal RNA gene 16S. And as for the Bacteroidetes, a probe taken from the 16S-RNA gene had been described in an article by Armougon Raoult in BMC Genomics 2008,9: 576, but without the primers, so that it was necessary to find primer sequences framing the known probe sequence that optimizes the sensitivity/specificity ratio, in other words, to find primer sequences flanking the probe sequence that are found in all the bacteria of the particular phylum Bacteroidetes and not found in the other phyla, particularly Firmicutes.

To accomplish this, the inventors searched in silico for regions of the gene that code for the portion 16S of ribosomal RNAs, enabling the amplification of nearly all of the Bacteroidetes on the one hand and the Firmicutes on the other hand, eliminating all risks of cross reactivity between the two groups. These different primer and probe systems being chosen in accordance with the experimental contingencies, the cross reactivities were tested in silico and then experimentally by using the DNA extracted from bacterial strains. The primer and probe systems adopted to recognize the Bacteroidetes do not experimentally recognize any other DNA coming from strains of Firmicutes and vice versa.

For the Lactobacillus target, the inventors chose the target on the gene tuf and a pair of primers and a probe were chosen in keeping with the technical constraints of the real-time PCR. From the DNA of 20 strains of Lactobacillus not recognized by the primer pair and the probe corresponding to the chosen molecular target, 20 sequences of the gene tuf not on deposit with the Genbank were amplified and sequenced, consensus sequences of primers and probes were chosen and tested on 96 DNA of Lactobacillus strains. This system of primers and probes that enables amplification of the maximum of Lactobacillus strains is preserved.

The sequences of Lactobacillus sp that were selected have a greater sensitivity and recognize a larger number of bacteria than those described in the prior art.

More particularly, one realizes a method of quantitative PCR amplification with the help of the following primers:
for said specific consensus sequence of the phylum Bacteroidetes:

sense primer:
SEQ. ID N^{o} 9 = 5′-AGCAGCCGCGGTAAT-3′,
antisense primer: SEQ. ID N^{o} 10:
5′-CTAHGCATTTCACCGCTAC-3′,

for said specific consensus sequence of bacteria of the genus Lactobacillus:

sense primer:
SEQ. ID N^{o} 13 = 5′- TACATYCCAACHCCAGAACG -3′,

where Y denotes C or T, H denotes A or C or T, and
antisense primer:

SEQ. ID N^{o} 14 = 5′ AAGCAACAGTACCACGACCA -3′.

for said specific consensus sequence of the phylum Firmicutes:

sense primer:
SEQ. ID N^{o} 17 = 5′- GTCAGCTCGTGTCGTGA-3′, et
antisense primer:
SEQ. ID N^{o} 18 = 5′-CCATTGTAKYACGTGTGT-3′

where K denotes G or T and Y denotes C or T.

More particularly again, one carries out real-time PCR amplification and quantification reactions with the help of specific hydrolysis probes chosen from among the following sequences:

1) for said specific sequence of the phylum Bacteroidetes:

SEQ. ID N^{o} 11 =
5′-GGGTTTAAAGGG-3′,

2) for said specific consensus sequence of bacteria of the genus Lactobacillus:

SEQ. ID N^{o} 15:
5′-AAGCCATTCTTRATGCCAGTTGAA-3′,

where R denotes A or G,

3) for said specific consensus sequence of the phylum Firmicutes:

SEQ. ID N^{o} 19:
5′-GTCAANTCATCATGCC-3′,

where N denotes either I, or one of A, T, C or G.

Said specific sequences thus detected and amplified correspond to:

1) a specific sequence of the ribosomal RNA gene 16S of Bacteroides fragilis,),

SEQ. N^{o} 12 =
5′-AGCAGCCGCGGTAATACGGAGGATCCGAGCGTTATCCGGATTTAT
TGGGTTTAAAGGGAGCGTAGGTGGACTGGTAAGTCAGTTGTGAAAGTT
TGCGGCTCAACCGTAAAATTGCAGTTGATACTGTCAGTCTTGAGTACA
GTAGAGGTGGGCGGAATTCGTGGTGTAGCGGTGAAATGCTTAG-3′

2) a specific consensus sequence of the bacteria Lactobacillus sp common to the bacteria Lactobacillus crispatus, Lactobacillus jensenii, Lactobacillus gasseri, Lactobacillus iners and Lactobacillus acidophilus, within the gene tuf coding for the elongation factor,

SEQ. ID N^{o} 16 = 5′-TACATCCCAACTCCAGAACGTGATACTGACAAGCCATTCTTAATGCCA
GTTGAAGACGTATTTACTATCACTGGTCGTGGTACTGTTGCTT -3′,

and

3) a specific consensus sequence taken from the ribosomal RNA gene 16S of Clostridium difficile of the phylum Firmicutes,

SEQ. ID N^{o} 20 = 5′-GTCAGCTCGTGTCGTGAGATGTTGGGTTAAGTCCCGCAACGAGCGCA
ACCCTTATTGTTAGTTGCCATCATTTAGTTGGGCACTCTAGCGAGACTGCCGGT
GACAAACCGGAGGAAGGTGGGGATGACGTCAAATCATCATGCCCCTTATGACCT
GGGCTACACACGTGCTACAATGG -3′.

The sense primer sequences and the complementary inverse sequences of the antisense primer are flanked and the probe sequences are flanked and italicized.

The above-described sequences SEQ. ID. No 1 to 20 are specified in the list of sequences appended to the present specification.

At the position corresponding to a nucleotide H, N, R, T or Y in the sequences SEQ. ID. No 10, 13, 15, 18 and 19 one finds, in the complementary target sequences, variable nucleotides as defined above.

The oligonucleotides of sequences SEQ. ID. No 10, 13, 15, 18 and 19 are thus used in fact in the form of equimolar mixtures of oligonucleotides of different sequences, said oligonucleotides of different sequences corresponding, for each sequence SEQ. ID. no 10, 13, 15, 18 and 19, to the different possible definitions of the respective sequences no 10, 13, 15, 18 and 19, namely:
an equimolar mixture of 3 different sequences for SEQ. ID. No 10 for which H is A, C and T, respectively,
an equimolar mixture of 2 different sequences for SEQ. ID. No 13 for which Y is C and T, respectively,
an equimolar mixture of 2 different sequences for SEQ. ID. No 15 for which R is A and G, respectively,
an equimolar mixture of 4 different sequences for SEQ. ID. No 18, for which KY are respectively GC, GT, TC, and TT,
an equimolar mixture of 4 different sequences for SEQ. ID. No 19 for which N represents A, T, C and G, respectively (when N represents one of A,T, C or G. On the other hand, when N is I (inosine), the oligonucleotide has a single sequence.)

These equimolar mixtures of oligonucleotides are obtained by using, during the oligonucleotide synthesis, equimolar mixtures of the different nucleotides involved.

More particularly, for the amplification of said specific sequence of the phylum Bacteroidetes, one performs the stage of hybridization of the primers and elongation at 48° C.

Preferably, one uses a large synthetic fragment of DNA serving as a quantification standard, this standard containing the specific sequences of the different molecular targets previously selected. The presence of several targets on the same fragment lets one quantify different targets in the same sample and co-quantify them in homogeneous manner by using the same range of standardization to detect different molecular targets, insofar as the constraints of the choice of the primers and the probe allow. This standardization range preserved in time is the guarantee of stability of the quantification system. This standardization range is diluted from 10⁷ to 1 copy of each of the molecular targets for 5 μl of DNA sample.

Advantageously, said concentrations Fi (bacteria of the phylum Firmicutes), Ba (bacteria of the phylum Bacteroidetes) or La (bacteria Lactobacillus sp) are determined by enzymatic amplification of real-time PCR type and quantification of the DNA of said DNA fragments of the bacteria of the phylum Firmicutes and Bacteroidetes and the genus Lactobacillus.

Preferably, one determines said concentrations of bacteria by enzymatic co-amplification of real-time PCR type of said DNA fragments of specific sequences respectively of the phylum Firmicutes and Bacteroidetes and the genus Lactobacillus contained on the one hand in said DNA extracted from the sample and on the other hand in synthetic DNA samples each containing said specific sequences of said bacteria serving as calibration standards for the DNA, said DNA fragments of specific sequences respectively of the phylum Firmicutes and Bacteroidetes and the genus Lactobacillus having a size of 70 to 150 nucleotides, preferably 90 to 120 nucleotides.

The detection and the quantification of said amplified fragments is done by means of marked probes of sequences distinct from those of the amplification primers and flanked by the latter, for each of said DNA fragments of specific sequences of the phylum Firmicutes and Bacteroidetes and the genus Lactobacillus, respectively, the markers of the different probes are markers which are different among themselves, particularly the known fluorescent markers of type VIC and FAM.

More particularly, said specific consensus sequences of said bacteria are taken from:
for the phylum Firmicutes: a fragment of the ribosomal RNA gene 16S of Clostridium difficile at positions 1026 to 1203, whose access number in the ribosomal bank RDP-II ((http://rdp.cme.msu.edu/) is S000260455.
for the phylum Bacteroidetes: the fragment from positions 537 to 721 of the ribosomal RNA gene 16S of Bacteroides fragilis whose access number in the ribosomal bank RDP-II is S000000037.
for the bacteria of the bacterial genus Lactobacillus, a sequence of 90 bases, common to the bacteria Lactobacillus crispatus, Lactobacillus jensenii, Lactobacillus gasseri, Lactobacillus ineri and Lactobacillus acidophilus within the gene tuf coding for the elongation factor at positions 253 to 343 of the reference gene GenBank AY 5621 91.1.
for the Archae Methanobrevibacter smithii, a sequence of 123 bases of the rRNA gene 16S specific to Archae Methanobrevibacter smithii at position 740 to 862 of the reference rRNA gene 16S Genbank CP000678.

By “probe” is meant here an oligonucleotide, preferably from 20 to 30 nucleotides, specifically hybridizing with said specific sequence and thus enabling its detection and its quantification in synthetic manner, thanks to measurement of the increase in fluorescence associated with the PCR reaction.

The probe makes it possible to detect the specific DNA amplified and to quantify it by comparing the signal strength with that of the quantification standard.

By “primer” is meant here an oligonucleotide of preferably 15 to 25 nucleotides that hybridizes specifically with one of the 2 terminal ends of the sequence that the DNA polymerase will amplify in the PCR reaction.

In all, the clinical results lead the inventors to estimate that a method for determination of the status of the intestinal flora of an individual advantageously involves the quantification of the two phyla Bactéroïdes and Firmicutes, on the one hand, and on the other hand bacteria of the genus Lactobacillus and of the species Methanobrevibacter smithii.

Thus, one performs the quantification of the aforesaid four so-called specific sequences of Methanobrevibacter smithii, the genus Lactobacillus, the phylum Bacteroidetes and preferably the phylum Firmicutes, respectively.

More preferably, one utilizes a large synthetic DNA fragment as a control standard for the DNA quantification, said large synthetic DNA fragment bringing together said specific sequences of each of said prokaryotic bacteria. The presence of several molecular targets on the same nucleic fragment makes it possible to quantify the different targets in the same sample and co-quantify them in homogeneous manner, the quantification making use of the same control range for several molecular species and enabling a comparison of the effectiveness of the different PCR reactions between themselves and from one assay to another over the course of time, avoiding the bias associated with the positive control.

More particularly, one uses a large synthetic DNA fragment serving as control standard for the DNA quantification, said large synthetic DNA fragment bringing together said specific sequences of Methanobrevibacter smithii, the genus Lactobacillus, phylum Bacteroidetes, and preferably phylum Firmicutes, respectively, more preferably inserted in a plasmid.

In the methods of DNA quantification for Real Time PCR it is important to know whether a positive reaction is due to a contamination by the recombinant plasmid used as the quantification standard or as positive control. To solve this problem, a cleavage site by a restriction enzyme is advantageously introduced into at least one of the molecular targets. This site being absent on the natural sequence. Thus, by enzymatic cleavage and analysis of the fragment amplified on gel agarose, or by using a PCR probe in real time that specifically recognizes the restriction site, one can thus detect the possible presence of the contaminating plasmid.

Thus, one carries out more particularly the following steps, in which:

1. one carries out an enzymatic amplification reaction of PCR type for the DNA of at least one so-called specific sequence of at least one of said agents, in the DNA extracted from said samples being tested according to the invention and in the DNA of the standard control sample, with the help of at least one set of primers able to amplify at least said authentic specific sequence and said modified specific sequence at the same time;

2. one verifies whether the amplifiates of the DNA extracted from said tested samples contain a so-called specific sequence, and

3. one detects the false positives resulting from possible contamination of said tested samples by the DNA coming from the control standard sample, by at least one of the following steps:

3a. one performs an enzymatic digestion of the PCR product obtained with an enzyme corresponding to the cleavage site and an assay on gel agarose of the digestion product, comparing this with the PCR product undigested by the restriction enzyme.

If the digested fragment comes from the amplification of the molecular target inserted in the control plasmid, it contains the restriction site, and it will be smaller in size than the undigested fragment.

3b. one performs a PCR type reaction in real time with direct and inverse primers of one of the molecular targets, and a specific probe for said exogenous sequence containing the restriction site.

Only a fragment coming from the control plasmid and containing the exogenous sequence could be amplified.

More advantageously, one performs the reactions of amplification and quantification by using sets of hydrolysis primers and probes specific to each of the different bacteria, so-called specific sequences of each of said bacteria being tested, and when necessary a specific human DNA sequence in the sample being tested, such as a specific sequence of human albumen, and said specific sequence contains a probe sequence flanked by sequences able to serve as primer in a PCR type amplification reaction of said specific sequences.

Again advantageously, one carries out a plurality of PCR enzymatic amplification reactions, simultaneous or not, of each of said specific sequences of said bacteria with the same large synthetic DNA control fragment, making use of a plurality of different sets of specific primers for each of said different specific sequences of each of said bacteria, the sequences of the different primers not overlapping between said different bacteria and said primers being able to be used in an enzymatic amplification reaction carried out by the same protocol and, in particular, at the same hybridization temperature.

One knows of various methods for constructing a large fragment of chimerical DNA combining several fragments, especially of different origin, particularly the method described in FR 2 882 063 where one prepares a large first fragment of synthetic double-stranded DNA of a particular sequence containing a series in a particular order of a plurality of n second small contiguous synthetic DNA fragments, essentially consisting in the dimerization of a plurality of n oligonucleotides by enzymatic amplification using a thermoresistant polymerase enzyme, involving:
a first stage of amplification reaction of nucleic acids of PCR type in presence of said polymerase enzyme, of a series of n oligonucleotides of particular sequences, without exogenous primers, comprising a series of cycles under temperature conditions enabling the hybridization of said oligonucleotides, followed by an elongation of the obtained complex, intended to place end to end in a particular order said oligonucleotides, the sequences of said oligonucleotides corresponding consecutively and alternately to the sense and antisense sequences of said different synthetic fragments, and each said oligonucleotide containing in its regions 5′ and 3′ complementary sequences to those of the following and preceding oligonucleotides, if so desired, and
a second stage of amplification using specific primers for the 5′ and 3′ terminal ends of said direct strand of said first large synthetic fragment being prepared, making it possible to produce identical copies of said first large fragment.

This technique is thus based on the use and the manipulation of a PCR artifact that consists in the hybridization of primers among themselves (dimerization of primers). This phenomenon is observed in the case when the PCR conditions, especially the temperature, are poorly adapted, and the primers contain partially complementary sequences.

The technique of construction thus involves selecting, from the target sequences, the sequences of oligonucleotides with an alternation of oligonucleotides of direct (“sense”) or inverse (also known as “reverse” or “antisense”) sequences. In order to make possible an end to end placement of these oligonucleotides, one takes care to introduce at position 3′ of the sequence of an oligonucleotide a complementary nucleotide sequence of the first nucleotides of the next oligonucleotide. These oligonucleotides will be hybridized by their complementary portions, and thanks to the polymerase activity, for example of Taq polymerase, a synthesis of 5′ at 3′ is realized in order to obtain double-strand fragments. The final (assembled) fragment is synthesized by PCR, using a pair of direct and inverse primers corresponding to the sequences of the terminal ends of the first desired large double-chain synthetic DNA fragment.

As mentioned above, advantageously said large synthetic DNA fragments are advantageously inserted into a plasmid.

This technique of genetic construction of a synthetic nucleotide fragment lets one place contiguously several molecular targets of interest. It is a simple method to carry out, quick and reliable, and does not require costly and burdensome equipment.

The present invention also deals with a diagnostic kit useful for carrying out a method of diagnostics and monitoring of the Firmicutes/Bacteroidetes ratio in stool samples according to the invention, characterized in that it comprises:
standard control DNA samples at a known concentration containing said specific sequences of each of said bacteria as defined above, and preferably a universal bacterial sequence as defined above, and again preferably a large synthetic DNA fragment bringing together said specific sequences of each of said bacteria as defined above, and preferably a universal bacterial sequence as defined above, as well as
said sets of specific primers of said specific modified synthetic DNA fragments of said bacteria and, again preferably, of said probes as defined above, and
reagents to carry out a PCR type DNA amplification reaction.

In one particular embodiment, one performs the quantification of said specific prokaryotic DNA of the bacteria Methanobrevibacter smithii, the bacterial genus Lactobacillus, the phylum Bacteroidetes and the phylum Firmicutes, to carry out the diagnostics and/or monitoring of the weight status of an individual.

The method of quantification according to the invention lets one correlate the monitoring of the weight status of a person with the monitoring of the contents of bacteria BA (Bacteroidetes), FI (Firmicutes), LA (Lactobacillus) and M. (Methanobrevibacter smithii), in particular as follows:
for patients undergoing a long-term antibiotic treatment, a decrease of BA (Bacteroidetes) and an increase of LA (Lactobacillus) represents a risk of weight gain, provided that LA (Lactobacillus) is greater than 10⁶.
for anorexic patients, a decrease in Methanobrevibacter smithii may be indicative of a favorable evolution of this pathology, spontaneously or under the effect of drug or nondrug therapy—for the obese under pharmaceutical treatment, an increase in BA (Bacteroidetes) and a decrease in LA (Lactobacillus) can be an indicator of good evolution of the treatment.

Finally, supplementation or specific treatments for the bacteria at issue may also contribute to the therapy of the persons involved, by trying to decrease specifically the level of M. (Methanobrevibacter smithii) in anorexics, and increase the level of BA (Bacteroidetes) and decrease the level of LA (Lactobacillus) in the obese or persons under long-term antibacterial treatment.

The present invention also provides a detection kit containing reagents to carry out a PCR type DNA amplification reaction and at least one set of primer oligonucleotides and preferably also at least one hydrolysis probe oligonucleotide useful for a detection and quantification by PCR amplification, preferably by Real Time PCR, comprising at least pairs of primer oligonucleotides able to amplify:
at least one specific sequence of Archae Methanobrevibacter smithii, and
at least one sequence chosen among:
   1) a specific consensus sequence of the phylum Bacteroidetes, and
   2) a specific consensus sequence of bacteria of the genus Lactobacillus, and
   3) a specific consensus sequence of the phylum Firmicutes.

More particularly, said kit contains at least one of the two pairs of primer oligonucleotides and preferably at least one of the hydrolysis probe oligonucleotides able to amplify at least one of said sequences of Methanobrevibacter smithii taken from the ribosomal RNA gene 16S and the rpoB gene, respectively, as follows:

a) the pair of primer oligonucleotides of sequences SEQ. ID No 2 and SEQ. ID No 3 and preferably the hydrolysis probe oligonucleotide of sequence SEQ. ID No 4, and

b) the pair of primer oligonucleotides of sequences SEQ. ID No 6 and SEQ. ID No 7, and preferably the hydrolysis probe oligonucleotide of sequence SEQ. ID No 8.

More particularly, a kit according to the invention contains the following pairs of primer oligonucleotides with preferably the hydrolysis probe oligonucleotides:

a) the pair of primer oligonucleotides of sequences SEQ. ID No 9 and SEQ. ID No 10, with preferably a hydrolysis probe oligonucleotide of sequence SEQ. ID No 11 for the amplification of a specific consensus sequence of the phylum Bacteroidetes and

b) the pair of primer oligonucleotides of sequences SEQ. ID N° 13 and SEQ. ID No 14, with preferably a hydrolysis probe oligonucleotide of sequence SEQ. ID No 15 for the amplification of a specific consensus sequence of bacteria of the genus Lactobacillus and

c) preferably the pair of primer oligonucleotides of sequences SEQ. ID No 17 and SEQ. ID No 18, with preferably a hydrolysis probe oligonucleotide of sequence SEQ. ID No 19 for the amplification of a specific consensus sequence of the phylum Firmicutes.

Even more particularly, a kit furthermore contains:
standard control DNA samples containing said specific sequences of Methanobrevibacter smithii, of bacteria of genus Lactobacillus, phylum Bacteroidetes, and preferably phylum Firmicutes, as defined above, and
preferably said large synthetic DNA fragment as defined above.

Preferably, a kit contains extraction reagents comprising at least one powderlike abrasive product, preferably glass powder, and a chemical and/or enzymatic lysis reagent.

The present invention also provides a method characterized in that, to carry out the extraction of the prokaryotic DNA of said stool sample, one performs the steps in which:

1) one prepares a homogeneous suspension of said stool sample in a buffer solution, preferably at a dilution of 50 to 150 g/l, and

2) one performs a mechanical lysis by mixing and agitation of said suspension from step 1 with an abrasive powderlike product, preferably glass powder, preferably washed in acid, and preferably one performs a stage of heating at 100° C. for 10 minutes, and

3) one performs a chemical and/or enzymatic lysis by mixing and agitation of the suspension obtained in step 2) with one or more chemical and/or enzymatic lysis buffers, then

4) one repeats step 2) with the mixture obtained in step 3), and

5) preferably one repeats step 3) with the mixture obtained in step 4).

In known manner, one finally separates the DNA either with known methods such as by fixation on a column containing silicate then washing to elute said DNA, separating it from the column, or by washing and centrifuging to recover said DNA fragments.

The performance of the first step of mechanical lysis lets one optimize and promote the work of chemical or enzymatic lysis agents in step 2) by promoting their penetration into the prokaryotic micro-organisms, enabling a partial degradation of the thick walls of the thick-walled prokaryotic microorganisms, and the total degradation of the thick walls is obtained only by at least a second mechanical lysis after said first chemical or enzymatic lysis.

In step 2), the heating to 100° C. enables finalization of the degradation of the components of the walls and membranes of the microorganisms.

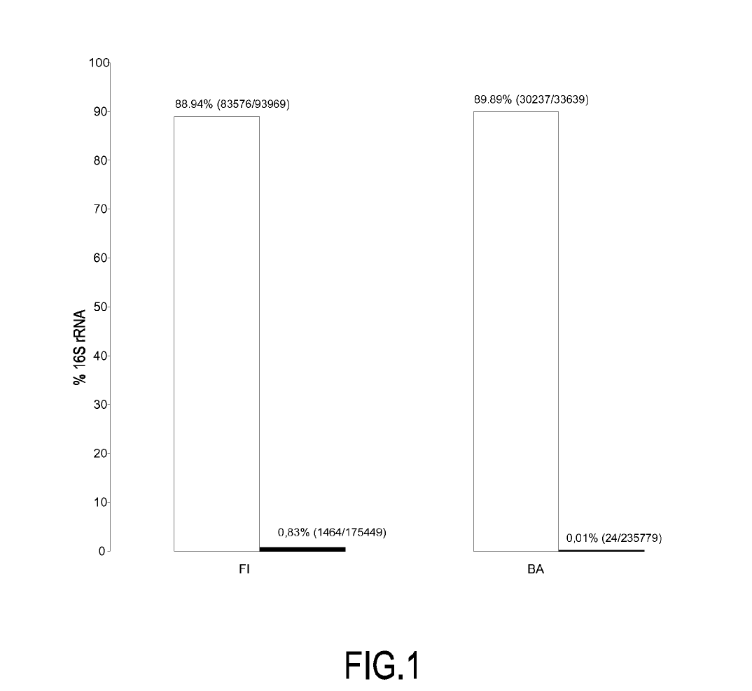

Other characteristics of the present invention will appear in the light of the following detailed description of various sample embodiments making reference to the sequence listing and FIGS. 1 to 4.

FIG. 1 shows along thee ordinate the percentage of ribosomal RNA 16S sequences and along the abscissa the results for the bacteria of the phylum Firmicutes (FI), and then the bacteria of the phylum Bacteroidetes (BA). The levels of sensitivity are represented by the first column in fine lines, namely, 88.94% for bacteria of the phylum Firmicutes and 89.89% for bacteria of the phylum Bacteroidetes. The levels of specificity are represented by the second column in bold, namely, 0.83% for bacteria of the phylum Firmicutes and 0.01% for bacteria of the phylum Bacteroidetes.

The values in brackets indicate the number of hybridizing sequences in the phylum studied (sensitivity) and outside the phylum studied (specificity).

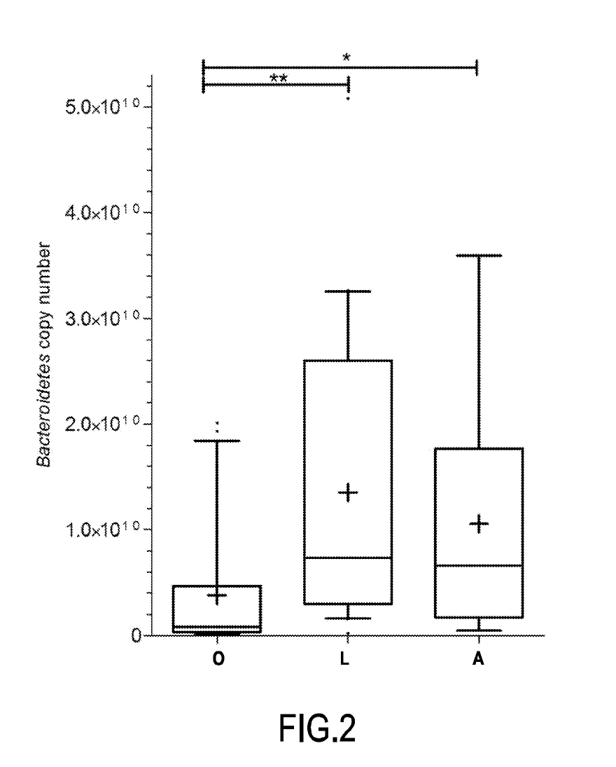

FIG. 2 shows the quantification of bacteria of the phylum Bacteroidetes in stools collected from three groups of obese individuals (O), control individuals (L) and anorexics (A).

The number of copies of bacteria Bacteroidetes plotted along the ordinate and the P value less than 0.05 is represented by the symbol “*” and the P value less than 0.01 is represented by “**”.

The cross indicates the average; the horizontal bar, the median; and the cube represents the distribution of values in the standard deviation.

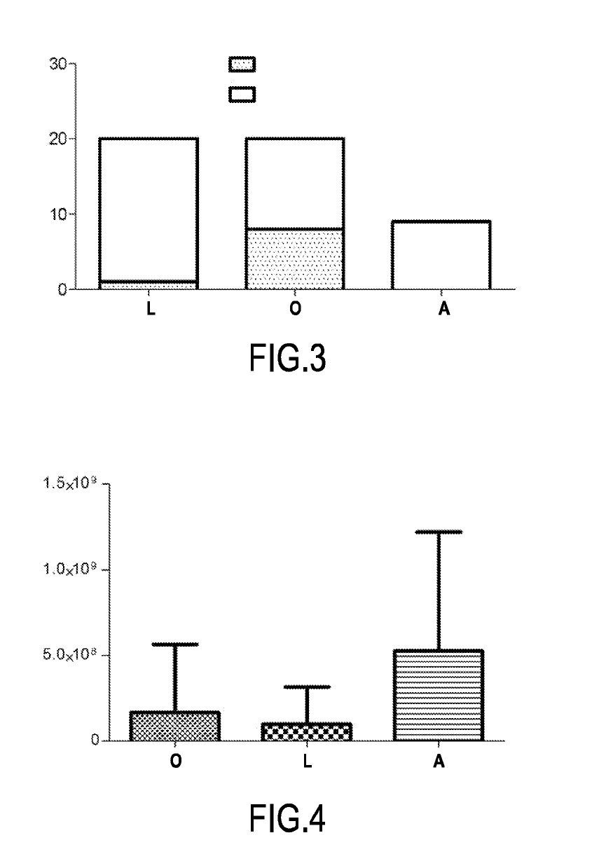

FIG. 3 shows the distribution of quantities greater than 10⁶ for number of bacteria of the genus Lactobacillus as detected and quantified by the method of the invention in the stools of anorexic (A), obese (O) and control (L) individuals.

The numbers along the ordinate represent the number of individuals.

In FIG. 3, [symbol] represents the number of copies of Lactobacillus above or equal to 10⁶, and [symbol] represents the number of copies of Lactobacillus below 10⁶.

FIG. 4 shows the quantification of Archea Methanobrevibacter smithii according to the method of the invention in stools of anorexic (A), obese (O) and control individuals (L).

FIG. 4 shows the standard deviation (height of the vertical bar) and the average (plateau of the cube), the numbers along the ordinate being the mean number of copies of Methanobrevibacter smithii.

### EXAMPLE NO 1

DNA extraction protocol according to the invention and comparison with the reference protocol published in the literature, for the detection of Archae Methanobrevibacter smithii in stools.

Archae Methanobrevibacter smithii is a methanogen, that is, an Archae able to transform the products of the digestion of food into methane, CH4. However, Methanosphaera stadtmanae is also a methanogenic Archae detected in stools. The inventors have counted on the fact that, given the importance of the biochemical reaction of methanogenesis to the intestinal physiology, Archae Methanobrevibacter smithii and not Methanosphaera stadtmanae was present and detectable in the stools of all individuals. This hypothesis was then verified and the inventors observed that Methanobrevibacter smithii was indeed detected in nearly all individuals by combining the detection of the ribosomal RNA gene 16S with that of the gene rpoB whereas Methanosphaera stadtmanae is only detected in less than 40% of individuals, making use of the same system. In order to test this hypothesis, the inventors conceived it would be necessary to lyse the wall of the Archae, in order to liberate the DNA of the Archae and make it accessible to molecular detection based on the PCR technique. For this, the inventors arrived at the following protocol by a series of trial and error. At a first stage, the inventors mechanically lysed the samples by agitation in presence of glass powder, followed by a chemical lysis, and then they extracted the DNA. The results showed that only 4/10 (40%) of the stool samples coming from 10 different individuals had a positive detection of the DNA of Methanobrevibacter smithii. To further improve the effectiveness of the protocol, the inventors then conceived of adding a second stage of mechanical lysis by glass powder and obtained a detection of the DNA of Methanobrevibacter smithii in 10/10 (100%) of the same stool samples. Likewise, the adding of a second stage of mechanical lysis surprisingly made it possible to increase from one to two log. the threshold of detection of positive samples by real-time PCR.

A protocol according to the invention is thus as follows:

1—around 500 mg of stool is suspended in 5 ml of Tris-HCL buffer, 0.05 M and pH 7.3, the mixture is homogenized by manual and vortex agitation until one gets a nearly homogeneous suspension;

2—250 μl of suspension is placed in a spiral tube of 1.5 ml containing 0.3 g (equivalent to a volume of around 10 μl, or less than 4% of the final volume) of glass powder whose particles measure less than 106 μm and washed in acid to eliminate fats, nucleic acids and DNAses and RNAses that might possibly result from the glass bead manufacturing processes (reference G4649, Sigma, Saint Quentin Fallavier, France) and agitated in the Fast Prep BIO 101 (Qbiogene, Strasbourg, France) at maximum speed (6.5) for 90 seconds;

3—the preparation is then heated to 100° C. for 10 minutes and then brought to room temperature. After this, a standard protocol of the Kit NucleoSpin® Tissue Mini Kit (Macherey Nagel, Hoerdt, France) is used as follows. One adds 180 μL of lysis buffer T1 and 25 μl of proteinase K to 20 mg/ml. The mixture is incubated at 56° C. over night;

4—one then performs a second mechanical lysis as described above;

5—one then performs a chemical lysis of known DNA lysis buffer, namely, 200 μl of buffer B3 (mixture in equal proportions of buffers B1 [Guanidine hydrochloride] and buffer B2 of composition available from the supplier) are added and the mixture is incubated for 10 minutes at 70° C., add 200 μl of ethanol, mix, apply to a silica column, centrifuge for 1 min at maximum speed, wash with 500 μl of buffer BW, centrifuge for 1 minute at maximum speed, wash with 600 μl of buffer B5 centrifuge for 1 minute at maximum speed;

6—one then deposits 100 μl of buffer BE, preheated to 70° C., at the center of the column and lets it incubate at room temperature for 1 to 2 minutes, then elutes the DNA by centrifugation for 1 minute at maximum speed. A negative extraction control of 250 μL of sterile water is introduced in each series of samples. The DNAs are preserved at −20° C. until their use. They will then be tested pure and diluted to 1/10 and 1/100 in order to reveal the possible presence of inhibitors of the PCR.

This phase of the process being finished, the inventors compared the protocol of the invention with the reference protocol for extraction of DNA of prokaryotes from stools. For this, the inventors collected 50 stool samples from 50 individuals having submitted stools for exploration of diarrhea. These 50 stool samples underwent a parallel DNA extraction by the protocol of the invention and by using the extraction kit QIAAMP Stool DNA mini kit (Qiagen, Courtaboeuf, France) as described in the literature [Eckburg P B. et Al. Diversity of the human intestinal microbial flora. Science 2005 Volume 308 page 1635-1638].

The principal stages of the reference DNA extraction method of the kit QIAAMP are:

1) dilution of the stool sample in a buffer of type PBS at pH=7.4 (2) and

2) incubation for one night at 56° C., in presence of proteinase K,

3) inactivation of proteinase K by heating to 70° C. for 10 minutes and

4) extraction of the total DNA by filtering on silicate column.

In this example, the inventors compared the number of copies of rRNA gene 16S and gene rpoB per ml of stool, using the two extraction protocols. The numerical data were analyzed using the non-parametric test of Kruskal-Wallis in the software EPIINFO version 3.4.1 (center sur dix controlant [for disease control?] and prevention, Atlanta, Ga.). The P values were used to show significant differences between the two methods and were calculated by using the nonparametric method of Kruskal-wallis for 2 groups. A P value less than 0.05 was taken to be significant.

Using the PCR quantification method marketed by Qiagen, the rDNA 16S gene of Methanobrevibacter smithii was detected in 44/50 (90%) of individuals and the gene rpoB was detected in 33/50 (66%) of individuals. Using the protocol of the invention as described in example 2, the ribosomal RNA gene 16S was detected by the same method of detection and quantification with the primers SEQ. ID. No. 6 and 7 and the probe SEQ. ID. No. 8 as described in example 2, in 50/50 (100%) of individuals, and the gene rpoB was detected in 49/50 (99%) of individuals with the primers SEQ. ID. No. 2 and 3 and the probe SEQ. ID. No. 4. Starting with one gram of stool, the extraction protocol of the invention was able to detect between 2 and 3 logarithms more DNA copies than the protocol marketed by Qiagen with a P value less than or equal to 0.00001 for the two genes analyzed. In this example, the negative controls remained strictly negative. These results show the superiority of the extraction protocol of the invention, as compared to an extraction protocol that is customarily used and marketed, both in number of individuals detected as positive and in quantity of DNA detected.

The quantification, in stools, of the ribosomal RNA genes 16S and rpoB of Archae Methanobrevibacter smithii, extracted by an extraction protocol according to the invention as compared to an extraction protocol marketed by QIAGEN (France) gives (in logarithm) the number of copies for each of the two genes.

1) Ribosomal RNA gene 16S,

Protocol of the Invention:

average value 2.05e¹⁰, standard deviation=6.13e⁺¹⁰, median=2.73e⁰⁶,

QIAGEN Extraction Protocol:

average value 2.66e⁰⁷, standard deviation=1.18e⁰⁸, median=8.98e⁰²,

P value=0.00001

2) Gene rpoB:

Extraction Protocol of the Invention:

average value 5.11e⁰⁹, standard deviation=1.48e⁺¹⁰, median=7.73e⁰⁵,

QIAGEN Extraction Protocol:

average value 6.67e⁰⁶, standard deviation=3.36e⁰⁷, median=4e⁰²,

P value=0.00001

For the quantification of M. smithii, the inventors used the primer sequences SEQ. ID. No. 2 and 3 and probe sequence SEQ. ID. No. 4 for the amplification of the ribosomal RNA gene 16S of M. smithii, and the primer sequences SEQ. ID. No. 6 and 7 and probe sequence SEQ. ID. No. 8 for the amplification of rpoB of M. smithii. The inventors established the following calibration curves for PCR quantification in the detection of M. smithii by amplification of the rRNA gene 16S and the gene rpoB, from known quantities of M. smithii obtained by culturing of M. smithii on agar-agar:

| | |
|---|---|
| 16S M. smithii | rpoB M. smithii |

| Ct | Quantity | Ct | Quantity |
|---|---|---|---|
| 20.27 | 1.00E+07 | 18.02 | 1.00E+07 |
| 22.99 | 1.00E+06 | 21.18 | 1.00E+06 |
| 26.79 | 1.00E+05 | 24.52 | 1.00E+05 |
| 29.72 | 1.00E+04 | 27.82 | 1.00E+04 |
| 30.52 | 1.00E+03 | 31.53 | 1.00E+03 |
| 32.36 | 1.00E+02 | 32.91 | 1.00E+02 |
| 33.84 | 1.00E+01 | 35.3 | 1.00E+01 |
| 35.03 | 1.00E+00 | 37.93 | 1.00E+00 |

A further study of 1500 stools coming from 1500 different individuals made it possible to determine a detection threshold of 10⁴ M. smithii organisms per ml of stool sample, based on the combined detection of the rRNA 16S and rpoB genes as described above. A concentration greater than 10⁴ M. smithii organisms per ml was detected in 97% of individuals. For the remaining 3%, the present detection threshold of 10⁴ organisms per ml of stool sample per ml was not reached.

### EXAMPLE NO 2

Specificity and sensitivity of the detection system for microorganisms belonging to the groups Firmicutes and Bacteroidetes in stools.

The determination of a real-time PCR system on the bacterial clades Firmicutes and Bacteroidetes required several months of perfecting and trial and error. The difficulties were both biological information and biotechnology. In fact, it was necessary for the inventors to perfect a technique of molecular detection of the two most numerically represented bacterial phyla as regards the number of ribosomal RNA 16S sequences in the electronic sequence banks. In particular, the phylum Firmicutes is represented by more than 94,000 sequences (or the phylum is the most important of the 70 phyla represented in RDP-II)[Cole J R, Chai B, Farris R J, Wang Q, Kulam-Syed-Mohideen A S, McGarrell D M et al.: The ribosomal database project (RDP-II): introducing myRDP space and quality controlled public data. Nucleic Acids Res 2007, 35: D169-D172] of ribosomal RNA 16S in the ribosomal database RDP-II. Likewise, the phylum Bacteroidetes is represented by more than 34,000 sequences (only the phylum Protebacteria is more important than the Bacteroidetes). To identify a system of 3 fragments of sequences (primers and probe) targeting nearly all of the species of the Firmicutes and Bacteroidetes respectively was a true challenge, especially since the system also had to be specific: namely, a minimum of species outside the phylum in question should not be detectable. These fragments of sequences having been determined, it was necessary to modify and adapt the experimental conditions so that the sensitivity of the real-time PCR reactions was optimal, and especially that there should be no cross detection between the different degenerated systems of primers and probe in question. The specificities were then tested by using the maximum DNA of reference bacterial strains at different concentrations. It was also necessary to suppress the parasitic fixations on the reaction medium due to the fact that one is qualifying bacteria that are present throughout the environment and in large quantity, without decreasing the sensitivity of the PCR reaction.

The difficulty associated in particular with the slight variability of the nucleic sequence of the ribosomal RNA gene 16S (many regions conserved) was therefore to identify PCR primers and a probe (1) in a region of limited size (at most 200 bases in order to be compatible with a system of real-time PCR detection), (2) with hybridization temperatures consistent with the system (at least an additional 10° C. for the melting point (Tm) of the probe as compared to that of the primers), (3) with nucleic sequences degenerated the least possible, so as to preserve a possibility of hybridization of the probe.

Taking as an example the real-time PCR system for the phylum Firmicutes, much perfecting was necessary. The probe or basic signature of the Glade Firmicutes, determined in silico, had at its core a series of undetermined bases (N): TCATGCCN[16]ACA. The inventors then attempted to elongate the pattern TCATGCC and obtain the specificity thus lost via the design of the primers and all of this in a region that needed to correspond to an amplification less than around 200 bases. Finally, the real-time PCR system created for the Glade Firmicutes is a quest for complementarity among the 3 elements (primers+probe). Taken individually, each of the sequences (primers and probe) is very sensitive to the Glade Firmicutes but not necessarily very specific. On the other hand, the merging of the 3 sequences of primers and probe SEQ. ID. No 17, No 18 and No 19 provides a great specificity and sensitivity for a use in real-time quantitative PCR.

Likewise, the three sequences of primers and probes SEQ. ID. No 9, No 10 and No 11 for the bacteria Bacteroidetes also provides a great specificity and sensitivity for a use in real-time quantitative PCR.

This specificity is demonstrated in this example, in which the sequence of the primers was synthesized by the Eurogentec company (Seraing, Belgium) and the sequence of the probes was synthesized by the Applied Biosystem company. The PCR amplification reactions were carried out on a MX3000™ system (Stratagene Europe, Amsterdam, Netherlands) making use of the kit QuantiTect PCR mix of the Qiagen company (Courtaboeuf, France) and 5 μmol of each primer and probe, 5 μl of DNA extracted from each of the 108 bacterial species reported in Table 1 after dilution to 1/10, 1/100, 1/1000, all of this under a final reaction volume of 25 μl. The real-time PCR program for the detection of Bacteroidetes involved: 95° C. 15 min, then 45 cycles of 95° C. 30 s, 48° C. 45 s, 72° C. 1 min), and for the detection of Firmicutes and Lactobacillus and Methanobrevibacter smithii: 95° C. 15 min, then 45 cycles of 95° C. 30 s, 60° C. 1 min. The concentrations of the quantities of probes and primers were adapted for each of the systems in order to preserve their effectiveness.

The results show that the system of detection of Bacteroidetes has a sensitivity of 89.89% since it detects 30,237 of the 33,639 ribosomal RNA 16S sequences of the bacteria of the phylum Bacteroidetes in the database RDP-II (FIG. 1) and that the system of detection of Firmicutes has a sensitivity of 88.94% since it detects 83,576 of the 93,969 sequences of the ribosomal RNA 16S gene of bacteria of the phylum Firmicutes of the database RDP-II. The use of the two detection systems Firmicutes and Bacteroidetes on the database RDP-II excluding these two phyla shows a false positive detection rate of 0.83% for Firmicutes and 0.01% for Bacteroidetes.

Likewise, the systems of the sequences SEQ. ID. No 2 and No 3 (primers) and No 4 (probe) for the ribosomal RNA 16S gene of Methanobrevibacter smithii and the sequences SEQ. ID. No 6 and No 7 (primers) and No 8 (probe) for the gene rpoB of Methanobrevibacter smithii, as well as the sequences SEQ. ID. No 13 and No 14 (primers) and No 15 (probe) for the sequences of the gene tuf of Lactobacillus spp. as previously described provide a great specificity (99%) and sensitivity (<50 copies) for a use in Real Time PCR.

The results of table 1 are expressed in CT, i.e., the number of cycles of amplification needed for the amplification to start, this CT value being related to the concentration of the nucleic product being quantified, namely, the lower the CT, the more elevated the concentration.

For the quantification, we constructed a calibration plasmid comprising a large synthetic hybrid DNA fragment containing the sequences SEQ. ID. No 1, No 2, No 12, No 16 and No 20, in the form of a double-strand DNA fragment constructed by amplification reaction, as described in FR-2 882 063.

We created a calibration plasmid range of 10⁷, one copy per well, the ten points of the plasmid range being tested for each amplification reaction.

### EXAMPLE NO 3

Analysis of bacterial flora and Archae by the method of the invention and weight condition of individuals.

Several works have shown that the composition of the digestive flora plays a role in obesity, including experimental works in the mouse [Samuel B S, Gordon J I: A humanized gnotobiotic mouse model of host-archaeal-bacterial mutualism. Proc Natl Acad Sci USA 2006, 103: 10011-10016; Ley R E, Backhed F, Turnbaugh P, Lozupone C A, Knight R D, Gordon J I: Obesity alters gut microbial ecology. Proc Natl Acad Sci USA 2005, 102: 11070-11075; Turnbaugh P J, Ley R E, Mahowald M A, Magrini V, Mardis E R, Gordon J I: An obesity-associated gut microbiome with increased capacity for energy harvest. Nature 2006, 444: 1027-1031]. In particular, it was shown that the Firmicutes/Bacteroidetes (F/B) ratio was associated with the obese phenotype or non-obese control, due to a reduction in the proportion of Bacteroidetes in obese persons, which is surprising because the bacteria Bacteroides thetaiotaomicron had been associated with an increase in obesity [Samuel B S, Gordon J I: A humanized gnotobiotic mouse model of host-archaeal-bacterial mutualism. Proc Natl Acad Sci USA 2006, 103: 10011-10016]. It was shown that, in the course of a weight-loss diet, the decrease in the F/B ratio of obese patients on the diet was correlated with weight loss, suggesting that the modulation of this ratio might constitute a therapeutic intervention [Ley R E, Turnbaugh P J, Klein S, Gordon J I: Microbial ecology: human gut microbes associated with obesity. Nature 2006, 444: 1022-1023]. The promoters of weight gain like the bacteria of the genus Lactobacillus might be implicated in these therapeutic interventions, their role having been shown in the fattening of farm animals [Khan M, Raoult D, Richet H, Lepidi H, La Scola B: Growth-promoting effects of single-dose intragastrically administered probiotics in chickens. Br Poult Sci 2007, 48: 732-735]. However, measurement of the F/B ratio is only being done at present by metagenomic methods, DNA chips and sequencing of large libraries of clones of the ribosomal DNA gene 16S, which are in the realm of research but not applicable for routine diagnostics. The inventors thus thought to use the invention to determine in reliable manner the F/B ratio by a real-time PCR technique, applicable on a routine basis. After favorable opinion of the Ethics Committee, the technique of quantification according to the invention was applied to a population of obese persons (17 to 72 years; body mass index BMI=47.09±10.66), 20 control persons (non-obese, non-anorexic; (13 to 68 years; BMI=20.68±2.014), and 9 persons having a psychological anorexia (19 to 36 years; BMI=12.73±1.602).

The results (Table 2) indicate that the quantification of Firmicutes is comparable for the three groups. The inventors thus observed that, contrary to the data published in the literature, the stools of obese persons have a smaller quantity of Bacteroidetes (FIG. 2) with a statistically significant difference between the obese and control group, on the one hand (** p<0.01), and between the anorexia and control group, on the other hand (*p<0.05). The number of Lactobacillus is higher in the obese persons, although the difference from the other two groups is not statistically significant. However, by using a threshold value of 10⁶ Lactobacillus, the difference in the number of Lactobacillus between obese and control persons (p=0.0197) and anorexics (p=0.0332) is significant (FIG. 3).

The systematic detection and quantification of Methanobrevibacter smithii has allowed the inventors to observe that the number of Methanobrevibacter smithii (FIG. 4) is higher in the obese than in the controls with an obese/control ratio of 1.72, but not in a statistically significant manner. On the other hand, the inventors observed, surprisingly, that the number of Methanobrevibacter smithii was more substantial in anorexics, by a factor of 3 and 5, respectively, as compared to the controls and the obese, and that the quantity of Methanobrevibacter smithii was significantly more substantial in anorexics than in the obese (p=0.0501). The level of Methanobrevibacter smithii is thus of interest to detect for a person suspected of anorexia. In all, the obese have a digestive flora poor in Bacteroidetes and rich in Lactobacillus. The flora of anorexics is similar to that of the controls for the quantities of Firmicutes, Bacteroidetes and Lactobacillus but contains more Methanobrevibacter smithii. The method of detection and quantification of the bacterial components of the microbiota used and proposed by the inventors enables a specific, dependable, rapid and repetitive detection and quantification of these four groups of microorganisms in a large number of stool samples. This method can be very easily used in current practice at the laboratories.

TABLE 1
List of microbial DNA used to determine the sensitivity/specificity
of the real-time PCRs for the detection of microorganisms belonging
to the groups Firmicutes and Bacteroidetes, in stools.

| | A | B |
|---|---|---|
| Prevotella bivia | 17.09 | >45 |
| Prevotella disiens | 16.7 | >45 |
| prevotella oulara | 16.45 | >45 |
| prevotella albensis | 19.31 | >45 |
| prevotella buccae | 18.64 | >45 |
| prevotella denticola | 16.69 | >45 |
| prevotella intermedia | 17.25 | >45 |
| prevotella nigrescens | 17.99 | >45 |
| prevotella melaninogenica | 16.3 | >45 |
| prevotella corporis | 15.18 | >45 |
| prevotella oris | 36.52 | >45 |
| Bacteroides fragilis | 20.37 | >45 |
| Bacteroides vulgatus | 20.36 | >45 |
| Bacteroides thetaiotaomicron | 21.27 | >45 |
| Bacteroides ovatus | 19.35 | >45 |
| alistipes finegoldii | 21.58 | >45 |
| alistipes putrenidis | 20.26 | >45 |
| captocytophaga sputigena | 18.56 | >45 |
| captocytophaga ochracea | 18.16 | >45 |
| captocytophaga ochracea | 18.18 | >45 |
| captocytophaga ochracae | 18.36 | >45 |
| captocytophaga haemolytica | 18.83 | >45 |
| captocytophaga granulosa | 18.62 | >45 |
| captocytophaga granulosa | 20.31 | >45 |
| captocytophaga gingivalis | 20.01 | >45 |
| captocytophaga haemolytica | 22.57 | >45 |
| captocytophaga cynodegmi | 17.15 | >45 |
| captocytophaga canimorsus | 23.04 | >45 |
| fusobacterium nucleatum | 38.6 | 18.36 |
| fusobacterium nucleatum | >45 | 21.86 |
| fusobacterium nucleatum | >45 | 22.15 |
| fusobacterium naviforme | 39.55 | 22.5 |
| fusobacterium nucleatum | >45 | 19.31 |
| fusobacterium nucleatum/naviforme | >45 | 18.54 |
| fusobacterium necrophorum | 38.15 | 22.61 |
| Bacillus cereus | >45 | 18.87 |
| Paenibacillus massiliensis | >45 | 19.1 |
| Paenibacillus timonensis | >45 | 20.73 |
| Paenibacillus sanguinis | >45 | 19.73 |
| Streptococcus gordonii | >45 | 19.49 |
| Streptococcus suis | >45 | 20.2 |
| Streptococcus vestibularis | >45 | 25.39 |
| Streptococcus pyogenes | >45 | 24.84 |
| Streptococcus peronis | >45 | 23.2 |
| Streptococcus infantis | >45 | 19.85 |
| Streptococcus cristatus | >45 | 23.28 |
| Streptococcus thermophilus | >45 | 21.02 |
| Streptococcus parasanguinis | >45 | 19.43 |
| Abiotrophia defective | >45 | 19.62 |
| Gemella sanguinis | >45 | 18.9 |
| Gemella bergeri | >45 | 20.48 |
| Enterococcus avium | >45 | 19.73 |
| Enterococcus gilvus | >45 | 22.2 |
| Enterococcus hirae | >45 | 21.56 |
| Enterococcus faecalis | >45 | 22.16 |
| Enterococcus raffinosus | 36.51 | 28.54 |
| Enterococcus casseliflavus | >45 | 23.38 |
| Enterococcus durans | >45 | 20.81 |
| Staphylococcus aureus | >45 | 25.59 |
| Corynebacterium accolens | >45 | 41.35 |
| Corynebacterium afermentans sub | >45 | 39.7 |
| afermentans | | |
| Corynebacterium afermentans sub | >45 | 39.99 |
| lipophilum | | |
| Corynebacterium amycolatum | >45 | 39.51 |
| Corynebacterium coyleae | >45 | 39.6 |
| Corynebacterium seminale | >45 | 32.28 |
| Corynebacterium urealyticum | 39.73 | 43.96 |
| Staphylococcus capitis | >45 | 22.53 |
| Staphylococcus arlettae | >45 | 21.47 |
| Staphylococcus hominis | >45 | 23.81 |
| Staphylococcus simulans | >45 | 21.61 |
| Staphylococcus caprae | >45 | 24.89 |
| Streptococcus mutans | 40.59 | 27.67 |
| Streptococcus oralis | >45 | 20.7 |
| Streptococcus salivarius | 38.27 | 28.67 |
| Streptococcus sanguinis | >45 | 26.3 |
| Streptococcus constellatus | >45 | 29.48 |
| Streptococcus mitis | >45 | 26.03 |
| Lactobacillus graminis | >45 | 29.99 |
| Lactobacillus acidophilus | >45 | 28.9 |
| Lactobacillus crispatus | >45 | 24.51 |
| Lactobacillus equi | >45 | 25.49 |
| Lactobacillus iners | >45 | 27 |
| Enterococcus faecium | >45 | 27.5 |
| Enterococcus faecium | >45 | 26.27 |
| Enterococcus faecalis | >45 | 17.23 |
| Bacillus odysseri | >45 | 23.8 |
| Bacillus neidei | >45 | 22.13 |
| Bacillus pycnus | >45 | 24.29 |
| Clostridium innocuum | >45 | 20.32 |
| Clostridium paraputrificum | >45 | 15.72 |
| Bacteroides uniformis | 15.47 | >45 |
| Bacteroides vulgatus | 16.52 | >45 |
| Bacteroides caccae | 15.17 | >45 |
| Clostridium beijerinckii | >45 | 18.63 |
| Clostridium putrificum | >45 | 16.69 |
| Clostridium septicum | >45 | 19.89 |
| Clostridium perfringens | >45 | 15.08 |
| Clostridium histolyticum | >45 | 16.33 |
| Clostridium difficile | >45 | 16.91 |
| Clostridium bifermentans | >45 | 22.04 |
| Clostridium thiosulforeducens | >45 | 14.21 |
| Clostridium saccharolyticum | >45 | 15.6 |
| Clostridium butyricum | >45 | 12.89 |
| Acidaminococcus fermentans | >45 | 20.15 |
| Acidaminococcus intestini | >45 | 25.65 |
| Parabacteroides distasonis | 9.62 | >45 |
| Peptostreptococcus anaerobius | >45 | 16.54 |
| Eubacterium saburreum | >45 | 24.06 |

A = DNA extracted from 108 strains tested by real-time PCR by the system Bacteroidetes.
B = DNA extracted from 108 strains tested by real-time PCR by the system Firmicutes.
(The results are given in Ct; the values shown bolded indicate the bacterial species for which there is a lack of specificity of the system).

TABLE 2
Quantification, according to the method of the invention, of
bacteria of the phyla Bacteroidetes, Firmicutes, Lactobacillus
and Archae Methanobrevibacter smithii in the stools of 49 control
individuals (lean), obese individuals (Ob) or those with an emaciation
associated with a phychological anorexia (ano).
Number of bacteria/Archae per g of stools

| Example | Bacteroidetes | Firmicutes | Lactobacillus | M. smithii |
|---|---|---|---|---|
| Lean1 | 3.26E+10 | 4.68E+10 | 0.00E+00 | 8.16E+07 |
| Lean2 | 5.08E+10 | 4.60E+10 | 0.00E+00 | 4.56E+08 |
| Lean3 | 2.91E+10 | 5.40E+10 | 0.00E+00 | 1.93E+04 |
| Lean4 | 1.62^{E}+10 | 1.52E+10 | 0.00E+00 | 2.55E+08 |
| Lean5 | 2.87^{E}+09 | 9.00E+09 | 0.00E+00 | 0.00E+00 |
| Lean6 | 6.12^{E}+09 | 1.49E+10 | 0.00E+00 | 2.77E+03 |
| Lean7 | 1.02^{E}+10 | 1.45E+10 | 9.64E+05 | 6.72E+07 |
| Lean8 | 6.80^{E}+09 | 1.52E+10 | 0.00E+00 | 0.00E+00 |
| Lean9 | 7.88^{E}+09 | 1.72E+10 | 0.00E+00 | 8.80E+08 |
| Lean10 | 5.20^{E}+09 | 3.46E+10 | 5.44E+07 | 5.72E+02 |
| Lean11 | 3.18^{E}+10 | 2.15E+10 | 4.80E+04 | 7.24E+05 |
| Lean12 | 1.58^{E}+09 | 2.14E+09 | 1.08E+04 | 2.96E+06 |
| Lean13 | 9.44^{E}+09 | 1.72E+10 | 0.00E+00 | 0.00E+00 |
| Lean14 | 1.67^{E}+10 | 4.32E+10 | 0.00E+00 | 0.00E+00 |
| Lean15 | 3.05^{E}+10 | 3.37E+10 | 0.00E+00 | 2.02E+08 |
| Lean16 | 2.15^{E}+09 | 1.70E+10 | 0.00E+00 | 0.00E+00 |
| Lean17 | 2.03^{E}+09 | 4.16E+09 | 0.00E+00 | 8.64E+06 |
| Lean18 | 4.72^{E}+09 | 1.39E+10 | 0.00E+00 | 6.68E+02 |
| Lean19 | 3.33^{E}+09 | 1.06E+10 | 0.00E+00 | 2.53E+06 |
| Lean20 | 2.06^{E}+08 | 4.68E+08 | 2.37E+04 | 9.16E+04 |
| Average | 1.35E+10 | 2.16E+10 | 2.77E+06 | 9.78E+07 |
| Ob1 | 1.93^{E}+10 | 3.82E+10 | 4.36E+08 | 3.62E+06 |
| Ob2 | 3.85^{E}+09 | 5.84E+10 | 3.36E+06 | 0.00E+00 |
| Ob3 | 1.77^{E}+08 | 3.80E+09 | 1.29E+06 | 1.96E+02 |
| Ob4 | 4.92^{E}+09 | 9.80E+09 | 0.00E+00 | 6.16E+08 |
| Ob5 | 2.01^{E}+10 | 2.44E+10 | 0.00E+00 | 0.00E+00 |
| Ob6 | 5.44^{E}+08 | 4.44E+09 | 0.00E+00 | 5.48E+03 |
| Ob7 | 1.05^{E}+10 | 1.50E+10 | 0.00E+00 | 3.15E+02 |
| Ob8 | 1.63^{E}+09 | 6.04E+09 | 4.28E+07 | 0.00E+00 |
| Ob9 | 1.94^{E}+09 | 7.20E+09 | 3.61E+08 | 4.68E+03 |
| Ob10 | 2.68^{E}+08 | 1.14E+10 | 2.21E+07 | 0.00E+00 |
| Ob11 | 9.08^{E}+08 | 8.84E+09 | 0.00E+00 | 1.01E+09 |
| Ob12 | 4.76^{E}+07 | 2.20E+09 | 5.28E+06 | 2.76E+04 |
| Ob13 | 2.14^{E}+09 | 1.12E+10 | 0.00E+00 | 1.40E+04 |
| Ob14 | 1.34^{E}+08 | 1.30E+10 | 0.00E+00 | 6.76E+02 |
| Ob15 | 6.20^{E}+09 | 2.14E+10 | 0.00E+00 | 5.44E+02 |
| Ob16 | 7.20^{E}+08 | 2.79E+09 | 3.96E+06 | 3.88E+02 |
| Ob17 | 6.60^{E}+08 | 6.44E+09 | 0.00E+00 | 1.14E+03 |
| Ob18 | 2.96^{E}+08 | 5.52E+09 | 1.96E+05 | 1.45E+09 |
| Ob19 | 3.37^{E}+08 | 6.28E+09 | 0.00E+00 | 2.82E+08 |
| Ob20 | 5.80^{E}+08 | 1.74E+10 | 0.00E+00 | 6.96E+05 |
| Average | 3.76E+09 | 1.37E+10 | 4.38E+07 | 1.68E+08 |
| Ano1 | 5.60^{E}+08 | 5.44E+09 | 5.56E+05 | 5.08E+03 |
| Ano2 | 4.72^{E}+08 | 6.72E+09 | 0.00E+00 | 1.17E+04 |
| Ano3 | 2.84^{E}+09 | 1.36E+10 | 2.30E+04 | 4.48E+08 |
| Ano4 | 2.44^{E}+10 | 1.98E+10 | 0.00E+00 | 3.82E+08 |
| Ano5 | 7.12^{E}+09 | 1.42E+10 | 0.00E+00 | 3.19E+03 |
| Ano6 | 3.59^{E}+10 | 1.48E+10 | 0.00E+00 | 9.40E+08 |
| Ano7 | 6.24^{E}+09 | 1.26E+10 | 0.00E+00 | 7.12E+02 |
| Ano8 | 1.09^{E}+10 | 2.04E+10 | 0.00E+00 | 8.84E+08 |
| Ano9 | 6.60^{E}+09 | 7.24E+09 | 1.45E+05 | 2.08E+09 |
| Average | 1.06E+10 | 1.28E+10 | 8.04E+04 | 5.26E+08 |

1. Method of detection and preferably of quantification of DNA, possibly comprising prokaryotic DNA, extracted from a stool sample of an individual, the method comprising controlling the quality of the extraction of the DNA by verifying whether a specific DNA of Methanobrevibacter smithii is detected and quantified at a rate of at least 10⁴ organisms of M. smithii/ml of said stool sample.

2. Method according to claim 1, characterized in that the DNA copy number of at least one DNA sequence specific for Methanobrevibacter smithii is quantified by quantitative PCR, chosen from among the following specific sequences:
a sequence taken from the gene taken from the 16S gene of the ribosomal RNA,
SEQ. ID. N^{o} 1 =
5′-CCGGGTATCTAATCCGGTTCGCGCCCCTAGCTTTCGTCCCTCACCG
TCAGAATCGTTCCAGTCAGACGCCTTCGCAACAGGCGGTCCTCCCAGGA
TTACAGAATTTCACCTCTACCCTGGGAG-3′,
and,
a sequence taken from the gene rpoB,
SEQ. ID. N^{o} 5 = 5′-AAGGGATTTGCACCCAACACAATTTGGTAAGATTTGTCCGAATGAAACCCC
AGAGGGTCCTAACTGTGGTC -3′

3. Method according to claim 2, characterized in that the number of copies of said DNA specific for Methanobrevibacter smithii is quantified by quantitative PCR in real time, involving PCR type enzymatic coamplification of a sequence specific for Methanobrevibacter smithii contained on the one hand in said DNA extracted from the stool sample and on the other hand in a synthetic sample of DNA fragments serving as a reference standard for quantification of the DNA, said sequence specific to Methanobrevibacter smithii being chosen among the following sequences or their complementary sequences:
a sequence of the RNA gene 16S amplifiable by the following primer sequences:
Sense primer, SEQ. ID. N^{o} 2:
5′-CCGGGTATCTAATCCGGTTC-3′,
and
Antisense primer, SEQ. ID. N^{o} 3:
5′-CTCCCAGGGTAGAGGTGAAA-3′,
and
a sequence of the gene rpoB amplified by the following sequence primers:
Sense primer, SEQ. ID. N^{o} 6:
5′-AAGGGATTTGCACCCAACAC-3′,
and
Antisense primers, SEQ. ID. N^{o} 7:
5′-GACCACAGTTAGGACCCTCTGG-3′.

4. Method according to claim 1, characterized in that at least two specific sequences of Methanobrevibacter smithii taken respectively from the ribosomal RNA gene 16S and from the gene rpoB are quantified.

5. Method according to claim 3, characterized in that one performs a reaction of amplification and quantification are carried out by PCR in Real Time, making use of specific hydrolysis probes for each of said specific sequences of Methanobrevibacter smithii, namely:
1) for the sequence of the ribosomal RNA gene 16S,
SEQ. ID N^{o} 4 = 5′-CCGTCAGAATCGTTCCAGTCAG -3′,
and
2) for the sequence of the gene rpoB,
SEQ. ID N^{o} 8 =
5′-ATTTGGTAAGATTTGTCCGAATG-3′,
and
a large fragment of synthetic DNA, serving as standard for quantification of the DNA, said large fragment of synthetic DNA bringing together said specific sequences with a plurality of samples of large synthetic DNA fragment of different known concentrations.

6. Method according to claim 1, characterized in that one also jointly quantifies in said DNA extracted from said stool sample at least one specific sequence chosen from among:
1) a specific consensus sequence of the phylum Bacteroidetes, and
2) a specific consensus sequence of bacteria of the genus Lactobacillus, and
3) a specific consensus sequence of the phylum Firmicutes.

7. Method according to claim 6, characterized in that a method of quantitative PCR amplification is carried out with the help of the following primers:
for said specific consensus sequence of the phylum Bacteroidetes:
sense primers: SEQ. ID N^{o} 9 =
5′-AGCAGCCGCGGTAAT-3′,
antisense primer: SEQ. ID N^{o} 10:
5′-CTAHGCATTTCACCGCTAC-3′,
for said specific consensus sequence of Lactobacillus spp.:
sense primer: SEQ. ID N^{o} 13 =
5′-TACATYCCAACHCCAGAACG-3′,
where Y denotes C or T, H denotes A or C or T, and
antisense primer:
SEQ. ID N^{o} 14 =
5′ AAGCAACAGTACCACGACCA-3′.
3′,
for said specific consensus sequence of the phylum Firmicutes:
sense primer: SEQ. ID N^{o} 17 =
5′-GTCAGCTCGTGTCGTGA-3′,
et
antisense primer: SEQ. ID N^{o} 18 =
5′-CCATTGTAKYACGTGTGT-3′
where K denotes G or T and Y denotes C or T.

8. Method according to claim 7, in which real-time PCR amplification and quantification reactions are carried out with the help of specific hydrolysis probes chosen from among the following sequences:
1) for said specific sequence of the phylum Bacteroidetes:
SEQ. ID N^{o} 11 =
5′-GGGTTTAAAGGG-3′
2) for said specific consensus sequence of Lactobacillus spp.:
SEQ. ID N^{o} 15:
5′-AAGCCATTCTTRATGCCAGTTGAA-3′,
where R denotes A or G.
3) for said specific consensus sequence of the phylum Firmicutes:
SEQ. ID N^{o} 19:
5′-GTCAANTCATCATGCC-3′,
where N denotes either I, or one of A, T, C or G.

9. Method according to claim 6, characterized in that the quantifications of the four said specific sequences of Methanobrevibacter smithii, the genus Lactobacillus, the phylum Bacteroidetes and the phylum Firmicutes, respectively, are carried out.

10. Method according to claim 9, characterized in that the quantification of said specific prokaryotic DNA of the bacteria Methanobrevibacter smithii, the bacterial genus Lactobacillus, the phylum Bacteroidetes and the phylum Firmicutes, are performed to carry out the diagnostics and/or monitoring of the weight status of an individual.

11. Method according to claim 1, characterized in that, to carry out the extraction of the prokaryotic DNA of said stool sample, the following steps are carried out in which:
1) a homogeneous suspension of said stool sample in a buffer solution at a dilution of 50 to 150 g/l is prepared, and
2) a mechanical lysis by mixing and agitation of said suspension from step 1 is carried out with an abrasive powderlike product, and a heating of the obtained suspension at 100° C. for 10 minutes, and
3) a chemical and/or enzymatic lysis is carried out by mixing and agitation of the suspension obtained in step 2) with one or more chemical and/or enzymatic lysis buffers, then
4) step 2) is repeated with the mixture obtained in step 3).

12. Detection kit useful to carry out a method according to claim 1, characterized in that it contains reagents to carry out a PCR type DNA amplification reaction and at least one set of primer oligonucleotides useful for a detection and quantification by PCR amplification comprising at least pairs of primer oligonucleotides able to amplify:
at least one specific sequence of Archae Methanobrevibacter smithii, and
at least one sequence chosen among:
1) a specific consensus sequence of the phylum Bacteroidetes, and
2) a specific consensus sequence of bacteria of the genus Lactobacillus, and
3) a specific consensus sequence of the phylum Firmicutes

13. Detection kit according to claim 12, characterized in that it contains at least one of the two pairs of primer oligonucleotides to amplify at least one of said sequences of Methanobrevibacter smithii taken from the ribosomal RNA gene 16S and the rpoB gene, respectively, as follows:
a) the pair of primer oligonucleotides of sequences SEQ. ID No 2 and SEQ. ID No 3, and
b) the pair of primer oligonucleotides of sequences SEQ. ID No 6 and SEQ. ID No 7.

14. Detection kit according to claim 12, characterized in that it contains the following pairs of primer oligonucleotides:
a) the pair of primer oligonucleotides of sequences SEQ. ID No 9 and SEQ. ID No 10 for the amplification of a specific consensus sequence of the phylum Bacteroidetes and
b) the pair of primer oligonucleotides of sequences SEQ. ID No 13 and SEQ. ID No 14, for the amplification of a specific consensus sequence of bacteria of the genus Lactobacillus and
c) the pair of primer oligonucleotides of sequences SEQ. ID No 17 and SEQ. ID No 18 for the amplification of a specific consensus sequence of the phylum Firmicutes.

15. Detection kit according to claim 12, characterized in that it contains extraction reagents comprising at least one powderlike abrasive product and a chemical and/or enzymatic lysis reagent.

16. Detection kit according to claim 13, characterized in that it contains at least one of the two pairs of primer oligonucleotides to amplify at least one of said sequences of Methanobrevibacter smithii taken from the ribosomal RNA gene 16S and the rpoB gene, respectively, as follows:
a) the pair of primer oligonucleotides of sequences SEQ. ID No 2 and SEQ. ID No 3, and
b) the pair of primer oligonucleotides of sequences SEQ. ID No 6 and SEQ. ID No 7.

17. Detection kit according to claim 14, characterized in that it contains the following pairs of primer oligonucleotides:
a) the pair of primer oligonucleotides of sequences SEQ. ID No 9 and SEQ. ID No 10, with a hydrolysis probe oligonucleotide of sequence SEQ. ID No 11 for the amplification of a specific consensus sequence of the phylum Bacteroidetes and
b) the pair of primer oligonucleotides of sequences SEQ. ID No 13 and SEQ. ID No 14, with a hydrolysis probe oligonucleotide of sequence SEQ. ID No 15 for the amplification of a specific consensus sequence of bacteria of the genus Lactobacillus and
c) the pair of primer oligonucleotides of sequences SEQ. ID No 17 and SEQ. ID No 18, with a hydrolysis probe oligonucleotide of sequence SEQ. ID No 19 for the amplification of a specific consensus sequence of the phylum Firmicutes.


SEQUENCE LISTING (20 sequences)

SEQ ID NO: 1 - DNA, 123 bp, Methanobrevibacter smithii
ccgggtatctaatccggttcgcgcccctagctttcgtccctcaccgtcagaatcgttccagtcagacgccttcgcaacaggcggtcctcccaggattacagaatttcacctctaccctgggag

SEQ ID NO: 2 - DNA, 20 bp, Methanobrevibacter smithii
ccgggtatctaatccggttc

SEQ ID NO: 3 - DNA, 20 bp, Methanobrevibacter smithii
ctcccagggtagaggtgaaa

SEQ ID NO: 4 - DNA, 22 bp, Methanobrevibacter smithii
ccgtcagaatcgttccagtcag

SEQ ID NO: 5 - DNA, 71 bp, Methanobrevibacter smithii
aagggatttgcacccaacacaatttggtaagatttgtccgaatgaaaccccagagggtcctaactgtggtc

SEQ ID NO: 6 - DNA, 20 bp, Methanobrevibacter smithii
aagggatttgcacccaacac

SEQ ID NO: 7 - DNA, 22 bp, Methanobrevibacter smithii
gaccacagttaggaccctctgg

SEQ ID NO: 8 - DNA, 23 bp, Methanobrevibacter smithii
atttggtaagatttgtccgaatg

SEQ ID NO: 9 - DNA, 15 bp, Bacteroides sp.
agcagccgcggtaat

SEQ ID NO: 10 - DNA, 19 bp, Artificial Sequence
  Sequence consensus of phylum Bacteroidetes
ctahgcatttcaccgctac

SEQ ID NO: 11 - DNA, 12 bp, Bacteroides sp.
gggtttaaaggg

SEQ ID NO: 12 - DNA, 184 bp, Bacteroides fragilis
agcagccgcggtaatacggaggatccgagcgttatccggatttattgggtttaaagggagcgtaggtggactggtaagtcagttgtgaaagtttgcggctcaaccgtaaaattgcagttgatactgtcagtcttgagtacagtagaggtgggcggaattcgtggtgtagcggtgaaatgcttag

SEQ ID NO: 13 - DNA, 20 bp, artificial sequence
  Sequence consensus of Lactobacillus sp.
tacatyccaachccagaacg

SEQ ID NO: 14 - DNA, 20 bp, Lactobacillus sp.
aagcaacagtaccacgacca

SEQ ID NO: 15 - DNA, 24 bp, artificial sequence
  Sequence consensus of Lactobacillus sp.
aagccattcttratgccagttgaa

SEQ ID NO: 16 - DNA, 91 bp, Lactobacillus sp.
tacatcccaactccagaacgtgatactgacaagccattcttaatgccagttgaagacgtatttactatcactggtcgtggtactgttgctt

SEQ ID NO: 17 - DNA, 17 bp, Clostridium difficile
gtcagctcgtgtcgtga

SEQ ID NO: 18 - DNA, 18 bp, artificial sequence
  sequence consensus of phylum Firmicutes
ccattgtakyacgtgtgt

SEQ ID NO: 19 - DNA, 16 bp, artificial sequence
  Sequence consensus of phylum Firmicutes
gtcaantcatcatgcc

SEQ ID NO: 20 - DNA, 178 bp, Clostridium difficile
gtcagctcgtgtcgtgagatgttgggttaagtcccgcaacgagcgcaacccttattgttagttgccatcatttagttgggcactctagcgagactgccggtgacaaaccggaggaaggtggggatgacgtcaaatcatcatgccccttatgacctgggctacacacgtgctacaatgg